(19) United States
(12) Patent Application Publication
(10) Pub. No.: US 2026/0207803 A1
(43) Pub. Date: Jul. 23, 2026

(54) SURFACE DISINFECTION IN CLOSED CHAMBER SYSTEMS

(71) Applicant: UVISAN LIMITED, Kingswood, Bristol (GB)

(72) Inventors: Jarek SALEK, Kingswood (GB); Luke FISHER, Kingswood (GB)

(73) Assignee: UVISAN LIMITED, Kingswood, Bristol (GB)

(21) Appl. No.: 19/141,548

(22) PCT Filed: Dec. 26, 2023
(86) PCT No.: PCT/IB2023/063253

§ 371 Date: Jun. 20, 2025

(30) Foreign Application Priority Data
Dec. 23, 2022 (GB) 2219719.8

(51) Int. Cl. A61L 2/104

(52) U.S. Cl. CPC A61L 2/104; A61L 2202/11; A61L 2202/121; A61L 2202/122; A61L 2202/123

(57) ABSTRACT

Apparatus for the sterilisation and/or disinfection of an object comprises a cabinet 1 for accommodating the object, means 29 within the cabinet for producing UV-C light, at least part of the internal surface of the cabinet being disrupted such that UV-C light is reflected therefrom in a diffused (or soft) manner so as to ensure that substantially the entire surface of the object is irradiated. The cabinet is provided with one or more doors 9 having an internally facing door panel with a perforated aluminium reflective surface 27.

### FIELD OF THE INVENTION

The invention is concerned with improvements in dispersion, efficacy and monitoring of UV-C light for the purpose of surface disinfection/sterilisation in closed chamber systems.

### BACKGROUND TO THE INVENTION

UV disinfection cabinets are known for disinfecting objects by means of ultraviolet radiation in the ‘C’ wavelength band (UV-C). Such a cabinet is used to kill microorganisms such as bacteria without a requirement for heat or chemicals. They comprise a cabinet with doors, and one or more UV-C lamps located within the cabinet.

### STATEMENTS OF THE INVENTION

According to the present invention, there is provided apparatus for sterilisation and/or disinfection of an object, the apparatus comprising a cabinet for accommodating the object, means within the cabinet for producing UV-C light, at least part of the internal surface of the cabinet being disrupted such that UV-C light is reflected therefrom in a diffused (or soft) manner so as to ensure that substantially the entire surface of the object is irradiated, the cabinet being provided with one or more doors having an internally facing door panel with a perforated aluminium reflective surface.

Having a disrupted internal surface increases the percentage of UV-C radiation reflected from the UV-C light producing means, dispersing the radiation at multiple angles from the uneven surface, thereby increasing the UV-C spread to a greater percentage of the cabinet surface so that the power of the reflected radiation to the surfaces of the object is increased.

Preferably, the internal surface is disrupted by being textured, eg, by being rippled, crazed or dimpled.

Providing at least one or more doors of the cabinet with an internally facing door panel with a perforated aluminium reflective surface both enhances the reflective power of the door panels and also allows users to see into the cabinet's interior during use.

Preferably, the means for producing UV-C light are recessed into an internal surface of the cabinet.

Preferably, the means within the cabinet for producing UV-C light is secured to the cabinet by means of a hidden fixing unit.

This aspect of the present invention reduces the proximity of the UV-C light producing means to the reflecting surface and maximise the percentage of the UV-C light reflected by the reflective surface.

Preferably, the cabinet is provided with at least one shelf for supporting said object, said shelf being an open wire support structure.

For instance, the shelf may be of the type known as etagere.

The open structure reduces the parts of the object in shadow and ensuring maximum coverage of the object by the UV-C radiation.

Preferably, said open wire structure is such that the object rests on a plurality of points of wire, thereby reducing the parts in shadow to a minimum.

More preferably, the open wire structure comprises spaced apart support wires having a triangle wave shape. This provides spaced apart apices on which rest objects placed in the cabinet.

### BRIEF DESCRIPTION OF THE DRAWINGS

The accompanying drawings are as follows:

FIG. 1 is a front view of apparatus in accordance with the present invention;

FIG. 2 is a side view of the apparatus of FIG. 1;

FIG. 3 is a perspective view of the apparatus of FIG. 1 with doors closed;

FIG. 4 is a perspective view of the apparatus of FIG. 1 with doors open;

FIGS. 5A to 5D are perspective, side, top and front views of a shelf of the apparatus of FIG. 1; and

FIG. 6 is a detailed perspective view from underneath of part of the apparatus of FIG. 1.

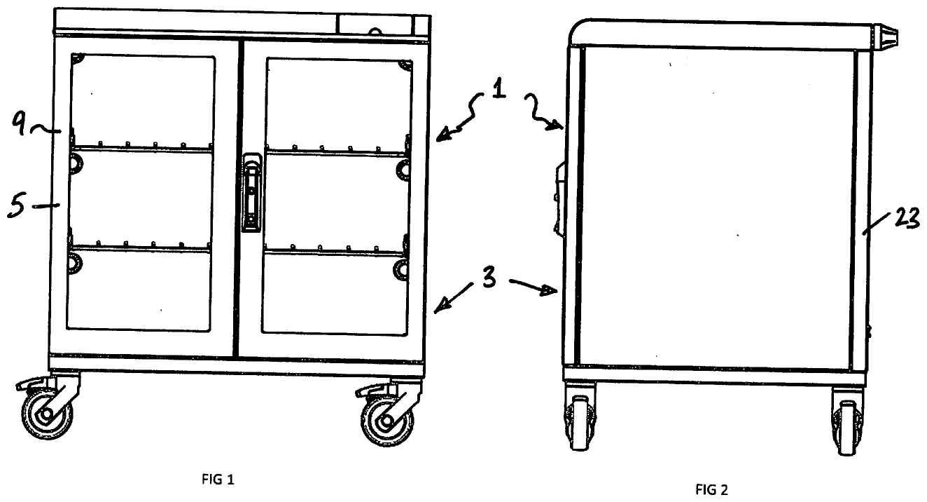

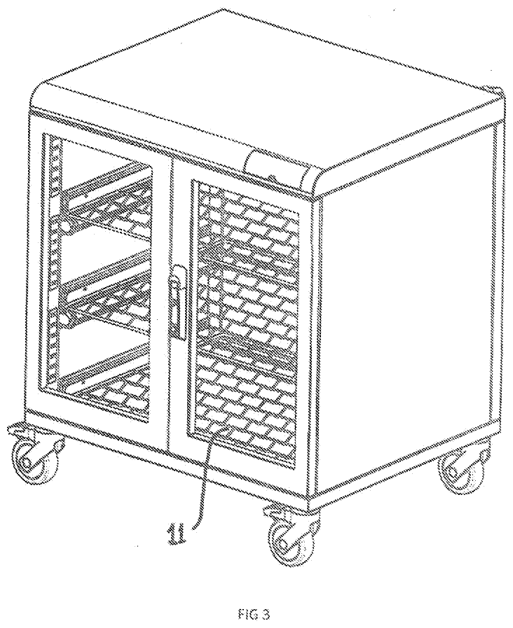

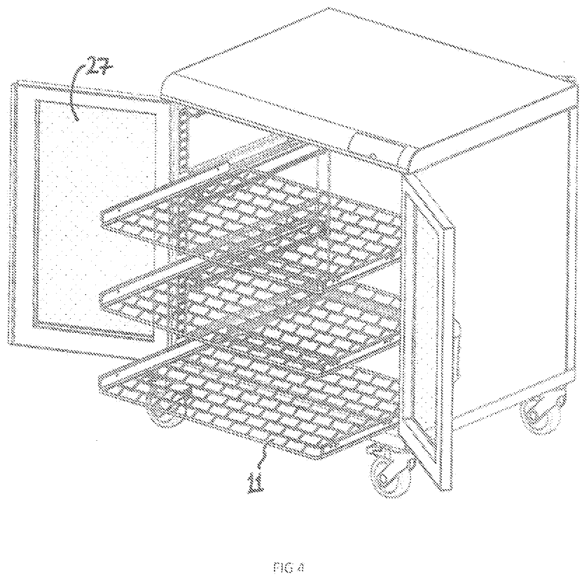

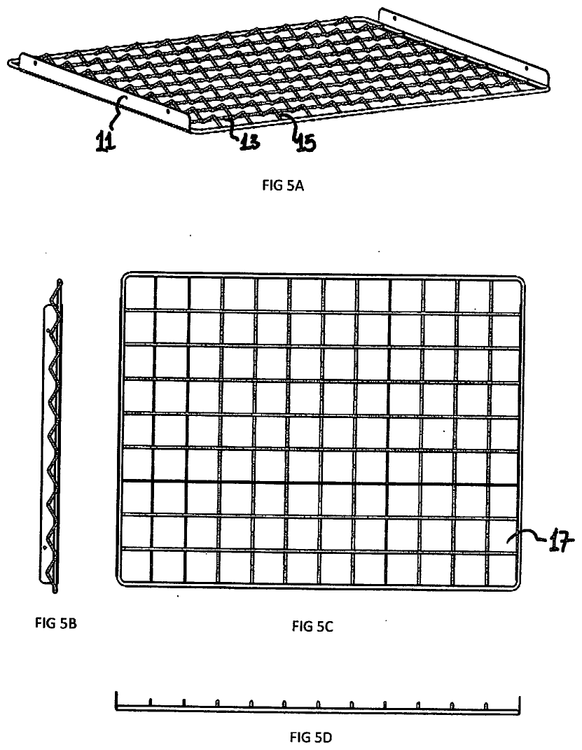

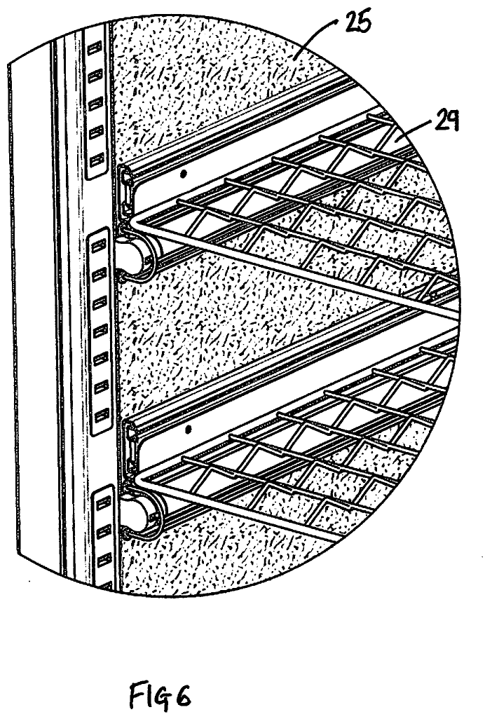

### DETAILED DESCRIPTION OF THE INVENTION

The present invention will now be described, by way of example only, with reference to the accompanying drawings.

Apparatus 1 for sterilisation and/or disinfection of an object comprises a cabinet 3 which includes a housing 5 mounted on wheels 7 and having a pair of doors 9 allowing access to the interior of the cabinet 3.

Mounted within cabinet 3, so as to be horizontally slidable into and out of cabinet 3, is a series of vertically spaced apart shelves 11. Each shelf 11 has an open wire support structure. Wires 13, which are spaced apart and extend from one side of the shelf to the other, are straight and wires 15, which are spaced apart and extend from the front to the rear of the shelf, have a triangle wave shape. Viewed from above, the wire structure defines an array of square spaces 17 and viewed from the side the wire structure shows the triangular wave shape, each triangle 19 providing an apex 21, a plurality of apices 21 supporting an object placed in cabinet 3. As a result, the UV-C light will, during operation of the apparatus 1, be able to reach virtually the entirety of the underside of the object.

The internal surfaces of the walls 23 of the chamber 3 are disrupted by being textured, eg, by being rippled, crazed or dimpled, as indicate at 25 of FIG. 6. As a result, a high percentage of UV-C radiation is reflected from the UV-C light producing means within cabinet 3, dispersing the radiation at multiple angles from the uneven surface, thereby increasing the UV-C spread to a greater percentage of the cabinet surface so that the power of the reflected radiation to these surfaces is increased.

Each of the doors 9 of the cabinet 3 is provided with an internally facing door panel with a perforated aluminium reflective surface having perforations 27. This feature provides both enhanced reflective power from the door panels and also allows users to see into the cabinet's interior during use.

Within cabinet 3 are mounted a plurality of UV-C light producing means, each comprising a UV-C light producing tube 29 located in one of the side walls of the cabinet 3 at a position just above an edge of a shelf 11.

1. Apparatus for sterilisation and/or disinfection of an object, the apparatus comprising a cabinet for accommodating the object, means within the cabinet for producing UV-C light, at least part of the internal surface of the cabinet being disrupted such that UV-C light is reflected therefrom in a diffused (or soft) manner so as to ensure that substantially the entire surface of the object is irradiated, the cabinet being provided with one or more doors having an internally facing door panel with a perforated aluminium reflective surface.

2. Apparatus according to claim 1, wherein said internal surface of the cabinet is textured.

3. Apparatus according to claim 2, wherein said internal surface of the cabinet rippled, crazed or dimpled.

4. Apparatus according to any of the preceding claims, wherein said means for producing UV-C light are recessed into an internal surface of the cabinet.

5. Apparatus according to any of claims 1 to 3, wherein the means within the cabinet for producing UV-C light is secured to the cabinet by means of a hidden fixing unit.

6. Apparatus according to any of claims 1 to 3, wherein the cabinet is provided with at least one shelf for supporting said object, said shelf being an open wire support structure.

7. Apparatus according to claim 6, wherein the open wire support structure is such that the object rests on a plurality of points of wire, thereby reducing the parts in shadow to a minimum.

8. Apparatus according to claim 6 or claim 7, wherein the open wire structure comprises spaced apart support wires having a triangle wave shape.
